(19) United States
(12) Patent Application Publication
(10) Pub. No.: US 2025/0276959 A1
(43) Pub. Date: Sep. 4, 2025

(54) SOLID STATE FORMS OF ZAVEGEPANT HYDROCHLORIDE AND PROCESS FOR PREPARATION THEREOF

(71) Applicant: TEVA CZECH INDUSTRIES S.R.O., Opava-Komarov (CZ)

(72) Inventors: Pavel Kolesa, Hlubocec (CZ); Zuzana Przeczkova, Ostrava (CZ); Adela Benysek-Bartova, Ostrava-Michalkovice (CZ)

(21) Appl. No.: 18/862,685

(22) PCT Filed: Nov. 9, 2023
(86) PCT No.: PCT/IB2023/061336

§ 371 Date: Nov. 4, 2024

Related U.S. Application Data
(60) Provisional application No. 63/451,951, filed on Mar. 14, 2023.
(60) Provisional application No. 63/423,836, filed on Nov. 9, 2022.

(51) Int. Cl. C07D 401/14

(52) U.S. Cl. CPC C07D 401/14

(57) ABSTRACT

The present disclosure encompasses solid state forms of Zavegepant hydrochloride, in embodiments crystalline polymorphs of Zavegepant hydrochloride, processes for preparation thereof, and pharmaceutical compositions thereof.

### FIELD OF THE DISCLOSURE

The present disclosure encompasses solid state forms of Zavegepant hydrochloride, in embodiments crystalline polymorphs of Zavegepant hydrochloride, processes for preparation thereof, and pharmaceutical compositions thereof.

### BACKGROUND OF THE DISCLOSURE

Zavegepant, (R)N-(3-(7-methyl-1H-indazol-5-yl)-1-(4-(1-methylpiperidin-4-yl)piperazin-1-yl)-1-oxopropan-2-yl)-4-(2-oxo-1,2-dihydroquinolin-3-yl)piperidine-1-carboxamide, has the following chemical structure.

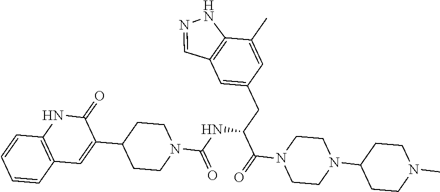 [SMILES: Cc1cc(C[C@@H](NC(=O)N2CCC(c3cc4ccccc4[nH]c3=O)CC2)C(=O)N2CCN(C3CCN(C)CC3)CC2)cc2cn[nH]c12]

Zavegepant is a third generation, high affinity, selective and structurally unique, small molecule CGRP receptor antagonist, and it is developed for acute treatment of migraine or COVID-19 respiratory infection.

The compound is described in International Publication No. WO2011/123232 and in Org. Process Res. Dev. 2012, 16, 1953-1966.

International Publication No. WO2022/217008 describes crystalline forms of Zavegepant. IPCOM000271435D describes crystalline forms of Zavegepant HCl.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new salt, solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional salts and solid state forms (including solvated forms) of Zavegepant.

### SUMMARY OF THE DISCLOSURE

The present disclosure provides crystalline polymorphs of Zavegepant hydrochloride, processes for preparation thereof, and pharmaceutical compositions thereof. These crystalline polymorphs can be used to prepare other solid state forms of Zavegepant hydrochloride, other Zavegepant salts and their solid state forms.

The present disclosure also provides uses of the said solid state forms of Zavegepant hydrochloride in the preparation of other solid state forms of Zavegepant hydrochloride or other salts and their solid state forms thereof.

The present disclosure provides crystalline polymorphs of Zavegepant hydrochloride for use in medicine, including for the acute treatment of migraine, for the prevention of migraine, or for COVID-19 respiratory infection.

The present disclosure also encompasses the use of crystalline polymorphs of Zavegepant hydrochloride of the present disclosure for the preparation of pharmaceutical compositions and/or formulations, particularly pharmaceutical compositions or formulations for oral or intranasal administration, and especially for intranasal administration. More particularly, the present disclosure encompasses the use of crystalline polymorphs of Zavegepant hydrochloride of the present disclosure for the preparation of pharmaceutical compositions or formulations particularly comprising solid Zavegepant hydrochloride, especially wherein the pharmaceutical compositions or formulations comprise solid Zavegepant hydrochloride. The pharmaceutical composition or formulation may be a solid dosage form (particularly for oral administration), especially tablets, powders, capsules, suppositories, sachets, troches, and lozenges; or may be in the form of a liquid (particularly for intravenous or intranasal, more particularly intranasal administration), especially a liquid, suspension, and elixir. Most preferably, the pharmaceutical composition or pharmaceutical formulation is a solution or suspension, particularly a solution or suspension for intranasal administration.

In another aspect, the present disclosure provides pharmaceutical compositions comprising crystalline polymorphs of Zavegepant hydrochloride according to the present disclosure. The pharmaceutical composition may particularly comprise solid Zavegepant hydrochloride, and may be a solid dosage form (particularly for oral administration), especially tablets, powders, capsules, suppositories, sachets, troches, and lozenges; or may be in the form of a liquid comprising solid Zavegepant hydrochloride (particularly for intravenous or intranasal, more particularly intranasal administration), especially liquid, suspension, and elixir. Most preferably, the pharmaceutical composition or pharmaceutical formulation is a suspension, particularly a suspension for intranasal administration. Pharmaceutical compositions according to any aspect of the present disclosure may include intranasal or oral dosage forms. Particularly, the pharmaceutical compositions may be an intranasal dosage form. More particularly, the pharmaceutical composition may be in the form of a liquid or a suspension, preferably a suspension, preferably for intranasal administration.

The present disclosure includes processes for preparing the above mentioned pharmaceutical compositions. The processes include combining any one or a combination of the crystalline polymorphs of Zavegepant hydrochloride with at least one pharmaceutically acceptable excipient. Particularly, the pharmaceutical compositions may comprise pharmaceutically acceptable excipient suitable for preparing an oral or intranasal dosage form, especially an intranasal dosage form. More particularly, the pharmaceutical compositions or formulations comprise solid Zavegepant hydrochloride. The pharmaceutical composition or formulation may be a solid dosage form (particularly for oral administration), especially tablets, powders, capsules, suppositories, sachets, troches, and lozenges; or may be in the form of a liquid (particularly for intravenous or intranasal, more particularly intranasal administration), especially a liquid, suspension, and elixir. Most preferably, the pharmaceutical composition or pharmaceutical formulation is a suspension, particularly a suspension for intranasal administration. More particularly, the pharmaceutical compositions may comprise one or more pharmaceutically acceptable excipients which are suitable for preparing a liquid or a suspension, preferably a suspension, for intranasal administration.

The crystalline polymorph of Zavegepant hydrochloride as defined herein and the pharmaceutical compositions or formulations of the crystalline polymorph of Zavegepant hydrochloride may be used as medicaments, such as for the acute treatment of migraine, for the prevention of migraine, or COVID-19 respiratory infection.

The present disclosure also provides methods of treating migraine, preventing migraine or treating COVID-19 respiratory infection, by administering a therapeutically effective amount of any one or a combination of the crystalline polymorphs of Zavegepant hydrochloride of the present disclosure, or at least one of the above pharmaceutical compositions, to a subject suffering from migraine or COVID-19 respiratory infection, or otherwise in need of the treatment.

The present disclosure also provides uses of crystalline polymorphs of Zavegepant hydrochloride of the present disclosure, or at least one of the above pharmaceutical compositions, for the manufacture of medicaments for treating e.g., migraine or COVID-19 respiratory infection, or for preventing migraine. The medicament may be administered as an intranasal dosage form, or may be administered as an oral dosage form, particularly an intranasal dosage form. More particularly, the medicament may be in the form of a liquid or a suspension, preferably a suspension, for intranasal administration.

According to any aspect or embodiment of the present disclosure, pharmaceutical compositions or formulations for the treatment of migraine or COVID-19 respiratory infection are preferably in the form of an intranasal dosage form (for example in the form of a liquid or a suspension for intranasal administration). According to any aspect or embodiment of the present disclosure, pharmaceutical compositions or formulations for the prevention of migraine are preferably in the form of an oral dosage form.

### BRIEF DESCRIPTION OF THE DRAWINGS

FIG. 1 shows a characteristic X-ray powder diffraction pattern (XRPD) of Zavegepant hydrochloride Form C1.

FIG. 2 shows a characteristic XRPD pattern of Zavegepant hydrochloride Form C2.

FIG. 3 shows a characteristic DSC thermogram of Zavegepant hydrochloride Form C1.

FIG. 4 shows a characteristic DSC thermogram of Zavegepant hydrochloride Form C2.

### DETAILED DESCRIPTION OF THE DISCLOSURE

The present disclosure encompasses solid state forms of Zavegepant hydrochloride, including crystalline polymorphs of Zavegepant hydrochloride, processes for preparation thereof, and pharmaceutical compositions thereof.

Solid state properties of Zavegepant hydrochloride and crystalline polymorphs thereof can be influenced by controlling the conditions under which Zavegepant and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Zavegepant hydrochloride described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Zavegepant hydrochloride. In some embodiments of the disclosure, the described crystalline polymorph of Zavegepant hydrochloride may contain from about 1% to about 20% (w/w), from about 5% to about 20% (w/w), or from about 5% to about 10% (w/w) of one or more other crystalline polymorph of the same Zavegepant hydrochloride.

Depending on which other crystalline polymorphs a comparison is made, the crystalline polymorphs of Zavegepant hydrochloride of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A compound may be referred to herein as chemically pure or purified compound or as substantially free of any other compounds. As used herein in this context, the expression “substantially free of any other compounds” will be understood to mean that the pure compound contains about 20% (w/w) or less, about 10% (w/w) or less, about 5% (w/w) or less, about 2% (w/w) or less, about 1% (w/w) or less, or about 0% of any other compound as measured, for example, by HPLC. Thus, pure or purified Zavegepant or Zavegepant hydrochloride described herein as substantially free of any compounds would be understood to contain greater than about 80% (w/w), greater than about 90% (w/w), greater than about 95% (w/w), greater than about 98% (w/w), greater than about 99% (w/w), or about 100% of the subject Zavegepant or Zavegepant hydrochloride. In some embodiments of the disclosure, the described pure or purified Zavegepant or Zavegepant hydrochloride may contain from about 1% to about 20% (w/w), from about 5% to about 20% (w/w), or from about 5% to about 10% (w/w) of one or more other compounds.

In specific embodiments, the above described pure or purified Zavegepant or Zavegepant hydrochloride may relate to enantiomeric purity, i.e., pure or purified Zavegepant or Zavegepant hydrochloride refers to Zavegepant or Zavegepant hydrochloride that is substantially free of enantiomers of Zavegepant or Zavegepant hydrochloride.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Zavegepant referred to herein as being characterized by graphical data “as depicted in” or “as substantially depicted in” a Figure will thus be understood to include any crystal forms of Zavegepant characterized with the graphical data having such small variations, as are well known to the skilled person, in comparison with the Figure.

As used herein, and unless stated otherwise, the term “anhydrous” in relation to crystalline forms of Zavegepant hydrochloride, relates to a crystalline form of Zavegepant hydrochloride, which does not include any crystalline water (or other solvents) in a defined, stoichiometric amount within the crystal. Moreover, unless otherwise indicated, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Zavegepant hydrochloride of the present disclosure corresponds to a crystalline polymorph of Zavegepant hydrochloride that is physically separated from the reaction mixture in which it is formed.

As used herein, unless stated otherwise, the XRPD measurements are taken using copper Kα radiation wavelength 1.54187 Å. XRPD peaks reported herein are measured using CuK α radiation, λ=1.54187 Å, typically at a temperature of 25±3° C.

As used herein, unless stated otherwise, solid state ¹³C NMR data is obtained using ¹³C CP/MAS NMR method. Particularly, as used herein, unless stated otherwise, the ¹³C CP/MAS NMR reported herein are measured at 500 MHz, preferably at a temperature of at 293 K±3° C. More particularly, as used herein, unless stated otherwise, the ¹³C CP/MAS NMR reported herein are measured at 500 MHz, preferably at a temperature of at 293 K±3° C. and at a magic angle spinning frequency ωᵣ/2π=11 kHz.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10 V) of a solvent. In this context, this expression would be understood to mean milliliters of the solvent per gram of the material being suspended, such that suspending 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

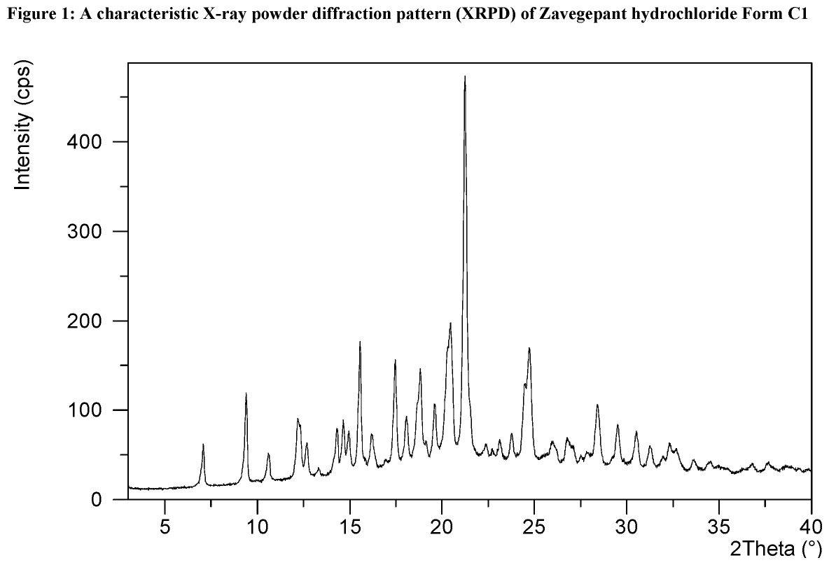

The present disclosure includes a crystalline polymorph of Zavegepant hydrochloride, designated Form C1. Crystalline Form C1 may be described by data selected from one or more of the following: an XRPD pattern having characteristic peaks at 7.1, 9.4, 15.6, 17.5 and 21.2 degrees 2-theta±0.2 degrees 2-theta, an XRPD pattern substantially as depicted in FIG. 1, and combinations thereof.

Crystalline Form C1 may be further by an XRPD pattern having characteristic peaks at 7.1, 9.4, 15.6, 17.5 and 21.2 degrees 2-theta±0.2 degrees 2-theta, and any one, two, three, four or five additional peaks at 10.6, 12.7, 14.7, 18.1 and 19.6 degrees 2-theta±0.2 degrees 2-theta.

Crystalline Form C1 may be described by an XRPD pattern having characteristic peaks at 7.1, 9.4, 10.6, 12.7, 14.7, 15.6, 17.5, 18.1, 19.6 and 21.2 degrees 2-theta±0.2 degrees 2-theta.

In any aspect or embodiment of the present disclosure, crystalline Form C1 of Zavegepant hydrochloride may be characterized by an X-ray powder diffraction pattern as described in any of the embodiments described herein, and wherein the X-ray powder diffraction pattern also has an absence of peaks at: 3.2 to 5.0 degrees 2-theta±0.2 degrees 2-theta, or 3.2 to 6.0 degrees±0.2 degrees 2-theta, or 3.2 to 6.3 degrees 2-theta±0.2 degrees 2-theta. Alternatively or additionally, according to aspect or embodiment of the present disclosure, crystalline Form C1 of Zavegepant hydrochloride may be characterized by an X-ray powder diffraction pattern as described in any of the embodiments described herein, and wherein the X-ray powder diffraction pattern also has an absence of peaks at: 7.6 to 8.2 degrees 2-theta±0.2 degrees 2-theta, or 7.6 to 8.6 degrees 2-theta±0.2 degrees 2-theta. Alternatively or additionally, according to aspect or embodiment of the present disclosure, crystalline Form C1 of Zavegepant hydrochloride may be characterized by an X-ray powder diffraction pattern as described in any of the embodiments described herein, and wherein the X-ray powder diffraction pattern also has an absence of peaks at: 9.9 to 10.1 degrees 2-theta±0.2 degrees 2-theta. Alternatively or additionally, according to aspect or embodiment of the present disclosure, crystalline Form C1 of Zavegepant hydrochloride may be characterized by an X-ray powder diffraction pattern as described in any of the embodiments described herein, and wherein the X-ray powder diffraction pattern also has an absence of peaks at: 11.1 to 11.7 degrees 2-theta±0.2 degrees 2-theta. Alternatively or additionally, according to aspect or embodiment of the present disclosure, crystalline Form C1 of Zavegepant hydrochloride may be characterized by an X-ray powder diffraction pattern as described in any of the embodiments described herein (particularly an XRPD pattern having characteristic peaks at 7.1, 9.4, 15.6, 17.5 and 21.2 degrees 2-theta±0.2 degrees 2-theta, and optionally any one, two, three, four or five additional peaks at 10.6, 12.7, 14.7, 18.1 and 19.6 degrees 2-theta±0.2 degrees 2-theta, or an XRPD pattern having characteristic peaks at 7.1, 9.4, 10.6, 12.7, 14.7, 15.6, 17.5, 18.1, 19.6 and 21.2 degrees 2-theta 0.2 degrees 2-theta; and also having an absence of peaks at any one, two, three, or four of (i)-(iv): (i) one of: 3.2 to 5.0 degrees 2-theta±0.2 degrees 2-theta, or 3.2 to 6.0 degrees±0.2 degrees 2-theta, or 3.2 to 6.3 degrees 2-theta±0.2 degrees 2-theta; (ii) one of: 7.6 to 8.2 degrees 2-theta±0.2 degrees 2-theta, or 7.6 to 8.6 degrees 2-theta±0.2 degrees 2-theta; (iii) 9.9 to 10.1 degrees 2-theta±0.2 degrees 2-theta; and (iv) 11.1 to 11.7 degrees 2-theta±0.2 degrees 2-theta.

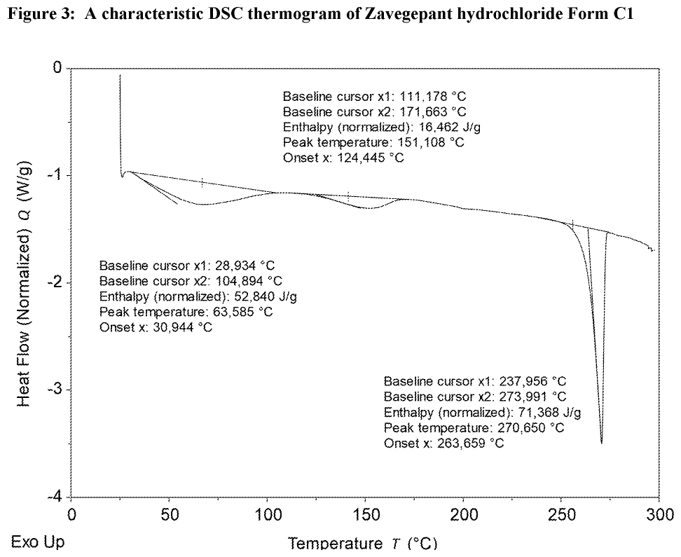

Typically, crystalline Form C1 DSC melting point is at about 264° C. (i.e., evaluated as onset point, measured by DSC 10° C./min). In some embodiments, Crystalline Form C1 of Zavegepant may be characterized by a DSC thermogram substantially as depicted in FIG. 3.

According to any aspect or embodiment of the present disclosure, crystalline Form C1 of Zavegepant hydrochloride is preferably isolated.

Crystalline Form C1 of Zavegepant hydrochloride according to any aspect or embodiment may be substantially free of any other crystalline forms of Zavegepant hydrochloride. In particular, the crystalline Form C1 of Zavegepant hydrochloride may contain: about 20% (w/w) or less, about 10% (w/w) or less, about 5% (w/w) or less, about 2% (w/w) or less, about 1% (w/w) or less, or about 0% of any other crystalline forms of Zavegepant hydrochloride, as measured, for example, by XRPD. Thus, crystalline Form C1 of Zavegepant hydrochloride described herein as substantially free of any other crystalline forms would be understood to contain greater than about 80% (w/w), greater than about 90% (w/w), greater than about 95% (w/w), greater than about 98% (w/w), greater than about 99% (w/w), or about 100% of Form C1 of Zavegepant hydrochloride.

According to any aspect or embodiment of the present disclosure, crystalline Form C1 of Zavegepant hydrochloride may be substantially free of amorphous Zavegepant hydrochloride. In particular, the crystalline Form C1 of Zavegepant hydrochloride may contain: about 20% (w/w) or less, about 10% (w/w) or less, about 5% (w/w) or less, about 2% (w/w) or less, about 1% (w/w) or less, or about 0% of amorphous Zavegepant hydrochloride, as measured, for example, by XRPD.

According to any aspect or embodiment of the present disclosure, the Zavegepant or Zavegepant hydrochloride, preferably Zavegepant hydrochloride Form C1, may be enantiomerically pure. By enantiomerically pure, it is meant that the Zavegepant or Zavegepant hydrochloride (preferably Zavegepant hydrochloride Form C1) is substantially free of enantiomers of Zavegepant or Zavegepant hydrochloride. In particular, in any aspect or embodiment of the present disclosure, the Zavegepant or Zavegepant hydrochloride (preferably Zavegepant hydrochloride Form C1) may be enantiomerically pure, and in particular contains: 20% (w/w) or less, about 10% (w/w) or less, about 5% (w/w) or less, about 2% (w/w) or less, about 1% (w/w) or less, or about 0% of other enantiomers of Zavegepant or Zavegepant hydrochloride respectively, as measured, for example, by HPLC.

Crystalline Form C1 of Zavegepant may be characterized by each of the above characteristics alone/or by all possible combinations, e.g., an XRPD pattern having peaks at 7.1, 9.4, 15.6, 17.5 and 21.2 degrees 2-theta±0.2 degrees 2-theta; an XRPD pattern as depicted in FIG. 1, and combinations thereof.

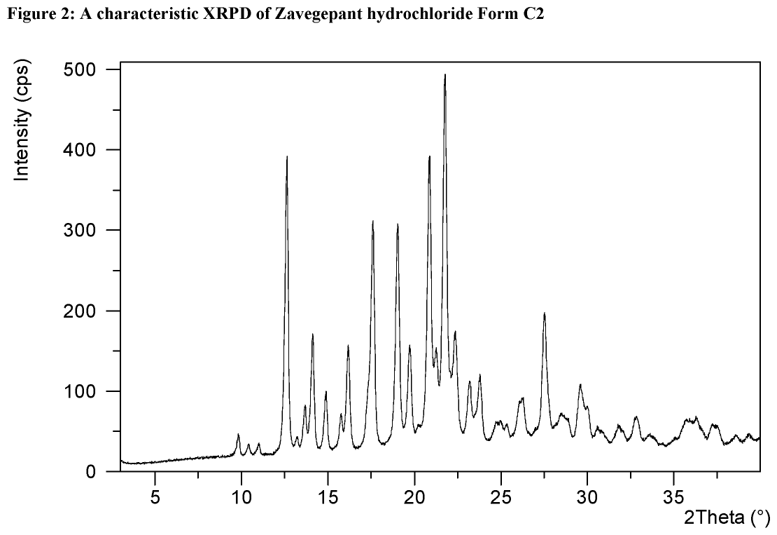

The present disclosure includes a crystalline polymorph of Zavegepant hydrochloride, designated Form C2. Crystalline Form C2 may be described by data selected from one or more of the following: an XRPD pattern having characteristic peaks at 12.6, 14.1, 14.9, 19.0, and 19.7 degrees 2-theta±0.2 degrees 2-theta; an XRPD pattern substantially as depicted in FIG. 2; and combinations thereof. Crystalline Form C2 may be further described by an XRPD pattern having characteristic peaks at 12.6, 14.1, 14.9, 19.0 and 19.7 degrees 2-theta±0.2 degrees 2-theta, and any one, two, three, four or five additional peaks at 9.8, 16.2, 17.6, 20.9 and 21.8 degrees 2-theta±0.2 degrees 2-theta.

Crystalline Form C2 may be described by an XRPD pattern having characteristic peaks at 9.8, 12.6, 14.1, 14.9, 16.2, 17.6, 19.0, 19.7, 20.9 and 21.8 degrees 2-theta 0.2 degrees 2-theta.

In any aspect or embodiment of the present disclosure, crystalline Form C2 of Zavegepant hydrochloride may be characterized by an X-ray powder diffraction pattern as described in any of the embodiments described herein, and wherein the X-ray powder diffraction pattern also has an absence of peaks at: 3.2 to 6.0 degrees 2-theta±0.2 degrees 2-theta, or 3.2 to 6.5 degrees 2-theta±0.2 degrees 2-theta, or 3.2 to 7.0 degrees±0.2 degrees 2-theta, or 3.2 to 7.5 degrees 2-theta±0.2 degrees 2-theta, or 3.2 to 8.0 degrees 2-theta±0.2 degrees 2-theta, or 3.2 to 8.5 degrees 2-theta±0.2 degrees 2-theta, or 3.2 to 9.0 degrees 2-theta±0.2 degrees 2-theta, or 3.2 to 9.3 degrees 2-theta±0.2 degrees 2-theta. Alternatively or additionally, according to aspect or embodiment of the present disclosure, crystalline Form C2 of Zavegepant hydrochloride may be characterized by an X-ray powder diffraction pattern as described in any of the embodiments described herein, and wherein the X-ray powder diffraction pattern also has an absence of peaks at: 11.5 to 12.1 degrees 2-theta±0.2 degrees 2-theta. Alternatively or additionally, according to aspect or embodiment of the present disclosure, crystalline Form C2 of Zavegepant hydrochloride may be characterized by an X-ray powder diffraction pattern as described in any of the embodiments described herein, and wherein the X-ray powder diffraction pattern also has an absence of peaks at: 16.7 to 16.8 degrees 2-theta±0.2 degrees 2-theta. Alternatively or additionally, according to aspect or embodiment of the present disclosure, crystalline Form C2 of Zavegepant hydrochloride may be characterized by an X-ray powder diffraction pattern as described in any of the embodiments described herein, and wherein the X-ray powder diffraction pattern also has an absence of peaks at: 18.1 to 18.5 degrees 2-theta±0.2 degrees 2-theta.

Alternatively or additionally, according to aspect or embodiment of the present disclosure, crystalline Form C2 of Zavegepant hydrochloride may be characterized by an X-ray powder diffraction pattern as described in any of the embodiments described herein (particularly an XRPD pattern having characteristic peaks at 12.6, 14.1, 14.9, 19.0 and 19.7 degrees 2-theta±0.2 degrees 2-theta and optionally any one, two, three, four or five additional peaks at 9.8, 16.2, 17.6, 20.9 and 21.8 degrees 2-theta±0.2 degrees 2-theta; or an XRPD pattern having characteristic peaks at 9.8, 12.6, 14.1, 14.9, 16.2, 17.6, 19.0, 19.7, 20.9 and 21.8 degrees 2-theta±0.2 degrees 2-theta) and also having an absence of peaks at any one, two, three, or four of (i)-(iv): (i) one of: 3.2 to 6.0 degrees 2-theta±0.2 degrees 2-theta, or 3.2 to 6.5 degrees 2-theta±0.2 degrees 2-theta or 3.2 to 7.0 degrees±0.2 degrees 2-theta, or 3.2 to 7.5 degrees 2-theta±0.2 degrees 2-theta, or 3.2 to 8.0 degrees 2-theta±0.2 degrees 2-theta, or 3.2 to 8.5 degrees 2-theta 0.2 degrees 2-theta, or 3.2 to 9.0 degrees 2-theta±0.2 degrees 2-theta, or 3.2 to 9.3 degrees 2-theta±0.2 degrees 2-theta; (ii) 11.5 to 12.1 degrees 2-theta±0.2 degrees 2-theta; (iii) 16.7 to 16.8 degrees 2-theta±0.2 degrees 2-theta; and (iv) 18.1 to 18.5 degrees 2-theta±0.2 degrees 2-theta. Alternatively, or additionally, crystalline Form C2 may be characterised by a solid state ¹³C NMR spectrum having peaks in the range of 200-100 ppm at: 170.2, 162.7, 158.4, 142.3, and 129.9 ppm±0.2 ppm; and/or a solid state ¹³C NMR spectrum having chemical shift absolute differences from reference peak at 123.5 ppm±1 ppm: 46.7; 39.2; 34.9; 18.8, and 6.5 ppm±0.1 ppm. Alternatively, according to any aspect or embodiment, crystalline Form C2 may be characterised by a solid state ¹³C NMR spectrum having peaks in the range of 200-100 ppm at: 170.2, 162.7, 158.4, 142.3, 139.2, 138.1, 135.1, 134.3, 131.1 and 129.9 ppm±0.2 ppm.

Alternatively, crystalline Form C2 according to any aspect or embodiment of the disclosure may be characterised by a solid state ¹³C NMR spectrum having peaks in the range of 200-100 ppm at: 170.2, 162.7, 158.4, 142.3, 139.2, 138.1, 135.1, 134.3, 131.1, 129.9, and 123.5 ppm±0.2 ppm, and additionally any one, two, three, or four peaks at: 122.1, 117.5, and 116.2 ppm±0.2 ppm. Alternatively, crystalline Form C2 may be described by a solid state ¹³C NMR spectrum having peaks in the range of 200-100 ppm at: 170.2, 162.7, 158.4, 142.3, 139.2, 138.1, 135.1, 134.3, 131.1, 129.9, 123.5, 122.1, 117.5, and 116.2 ppm 0.2 ppm.

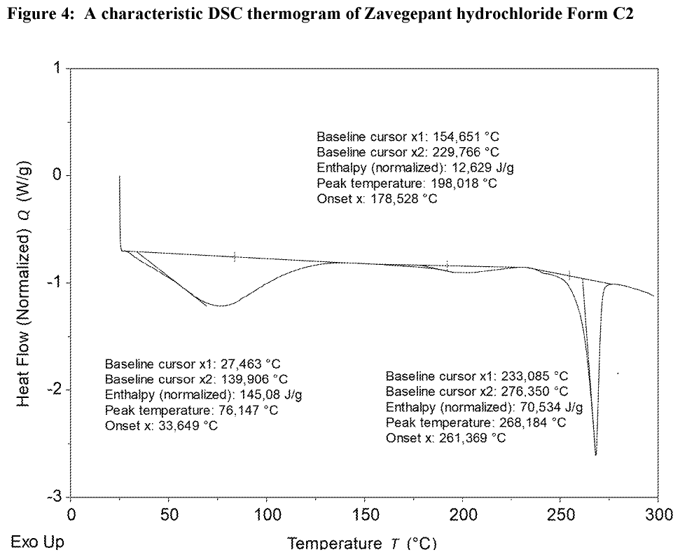

Typically, crystalline Form C2 DSC melting point is at about 261° C. (i.e., evaluated as onset point, measured by DSC 10° C./min). In some embodiments, Crystalline Form C2 of Zavegepant may be characterized by a DSC thermogram substantially as depicted in FIG. 4.

According to any aspect or embodiment of the present disclosure, crystalline Form C2 of Zavegepant hydrochloride is preferably isolated.

Crystalline Form C2 of Zavegepant hydrochloride may be an anhydrous form.

Crystalline Form C2 of Zavegepant hydrochloride according to any aspect or embodiment may be substantially free of any other crystalline forms of Zavegepant hydrochloride. In particular, the crystalline Form C2 of Zavegepant hydrochloride may contain: about 20% (w/w) or less, about 10% (w/w) or less, about 5% (w/w) or less, about 2% (w/w) or less, about 1% (w/w) or less, or about 0% of any other crystalline forms of Zavegepant hydrochloride, as measured, for example, by XRPD. Thus, crystalline Form C2 of Zavegepant hydrochloride described herein as substantially free of any other crystalline forms would be understood to contain greater than about 80% (w/w), greater than about 90% (w/w), greater than about 95% (w/w), greater than about 98% (w/w), greater than about 99% (w/w), or about 100% of Form C2 of Zavegepant hydrochloride.

According to any aspect or embodiment of the present disclosure, crystalline Form C2 of Zavegepant hydrochloride may be substantially free of amorphous Zavegepant hydrochloride. In particular, the crystalline Form C2 of Zavegepant hydrochloride may contain: about 20% (w/w) or less, about 10% (w/w) or less, about 5% (w/w) or less, about 2% (w/w) or less, about 1% (w/w) or less, or about 0% of amorphous Zavegepant hydrochloride, as measured, for example, by XRPD.

According to any aspect or embodiment of the present disclosure, the Zavegepant hydrochloride Form C2, may be enantiomerically pure. By enantiomerically pure, it is meant that the Zavegepant hydrochloride Form C2 is substantially free of enantiomers of Zavegepant hydrochloride. In particular, in any aspect or embodiment of the present disclosure, the Zavegepant hydrochloride Form C2 may be enantiomerically pure, and in particular contains 20% (w/w) or less, about 10% (w/w) or less, about 5% (w/w) or less, about 2% (w/w) or less, about 1% (w/w) or less, or about 0% of other enantiomers of Zavegepant hydrochloride respectively, as measured, for example, by HPLC.

Crystalline Form C2 of Zavegepant hydrochloride may be characterized by each of the above characteristics alone/or by all possible combinations, e.g., an XRPD pattern having peaks at 12.6, 14.1, 14.9, 19.0 and 19.7 degrees 2-theta±0.2 degrees 2-theta; an XRPD pattern as depicted in FIG. 2, and combinations thereof.

Crystalline Form C2 of Zavegepant hydrochloride may have one or more advantageous properties, as discussed herein above. For example, Form C2 is stable for a period of at least for 6 months at temperature of about 25° C. and a relative humidity (“RH”) of 60% RH, and at a temperature of about 40° C. and 75% RH.

The above crystalline polymorphs can be used to prepare other crystalline polymorphs of Zavegepant hydrochloride, other Zavegepant salts and their solid state forms. Solid state forms may be crystalline polymorphs, co-crystals and complexes of Zavegepant or of Zavegepant salt.

The present disclosure encompasses a process for preparing other solid state forms of Zavegepant hydrochloride and their solid state forms thereof. The process includes preparing any one of the crystalline polymorphs of Zavegepant hydrochloride by the processes of the present disclosure. The process may further comprise converting said crystalline polymorph of Zavegepant hydrochloride to other crystalline polymorph of Zavegepant or to other Zavegepant salt.

The present disclosure provides the above described crystalline polymorphs of Zavegepant hydrochloride for use in the preparation of pharmaceutical compositions comprising Zavegepant hydrochloride and/or crystalline polymorphs thereof.

The present disclosure also encompasses the use of crystalline polymorphs of Zavegepant hydrochloride of the present disclosure for the preparation of pharmaceutical compositions of crystalline polymorph Zavegepant hydrochloride and/or crystalline polymorphs thereof. Particularly, the pharmaceutical compositions may be used for intranasal or oral administration, and more particularly for intranasal administration.

The present disclosure includes processes for preparing the above mentioned pharmaceutical compositions. The processes include combining any one or a combination of the crystalline polymorphs of Zavegepant hydrochloride of the present disclosure with at least one pharmaceutically acceptable excipient. Particularly, the pharmaceutical compositions may comprise pharmaceutically acceptable excipient suitable for making formulation of intranasal or oral administration, and more particularly for intranasal administration. Pharmaceutical combinations or formulations of the present disclosure contain any one or a combination of the solid state forms of Zavegepant hydrochloride of the present disclosure. In addition to the active ingredient, the pharmaceutical formulations of the present disclosure can contain one or more excipients. Excipients are added to the formulation for a variety of purposes. For example, excipients may be added to assist in formation of formulation suitable for intranasal administration.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., Klucel®), hydroxypropyl methyl cellulose (e.g., Methocel®), liquid glucose, magnesium aluminum silicate, maltodextrin, methylcellulose, polymethacrylates, povidone (e.g., Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Zavegepant and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, intranasal and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Zavegepant hydrochloride can be administered. For example, it can be administrated intranasally or orally. Zavegepant hydrochloride may be formulated for administration to a mammal, in embodiments to a human, by injection. Zavegepant hydrochloride can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Zavegepant hydrochloride and the pharmaceutical compositions and/or formulations of Zavegepant hydrochloride of the present disclosure can be used as medicaments, in embodiments in the treatment of migraine or COVID-19 respiratory infection. The medicament may preferably be administrated in intranasal or oral form, and more preferably in intranasal form.

The present disclosure also provides methods of treating migraine or COVID-19 respiratory infection by administering a therapeutically effective amount of any one or a combination of the crystalline polymorphs of Zavegepant hydrochloride of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Powder X-ray Diffraction was performed on an X-Ray powder diffractometer PanAlytical X'pert Pro; CuKα radiation (λ=1.54187 Å); X'Celerator detector with active length 2.122 degrees 2-theta; laboratory temperature 25±3° C.; zero background sample holders. Prior to analysis, the samples were gently ground using a mortar and pestle to obtain a fine powder. The ground sample was adjusted into a cavity of the sample holder and the surface of the sample was smoothed using a cover glass.

### Measurement Parameters

Scan range: 3-40 degrees 2-theta
Scan mode: continuous
Step size: 0.0167 degrees (or 0.0131 for form C2)
Step size: 42 s (or 41 s for form C2)
Sample spin: 60 rpm
Sample holder: zero background silicon plate

### Differential Scanning Calorimetry (“DSC”) Method

### Instrument: TA Discovery

Method: heating rate 10° C./min, temperature range 25° C.-300° C., nitrogen flow 50 ml/min, aluminum Tzero pans with Tzero hermetic lids, 40 μl.

### ¹³C-Cp/Mas Nmr Method:

Spectra were measured at 11.7 T using a Bruker Avance III HD 500 US/WB NMR spectrometer (Karlsruhe, Germany, 2013) with a 4-mm probehead. The ¹³C CP/MAS NMR spectra were recorded at room temperature and the frictional heating of the spinning samples was compensated. The ¹³C CP/MAS NMR spectra employing cross-polarization were acquired using the standard cross-polarization pulse scheme at spinning frequency of 11 kHz. The dipolar decoupling SPINAL64 is applied during the data acquisition. The ¹³C scales are referenced to glycine (176.03 ppm for ¹³C).

The NMR spectrometer was calibrated and all experimental parameters are carefully optimized prior the recording of the spectra. Magic angle is set using KBr during the standard optimization procedure and homogeneity of magnetic field is optimized using adamantine sample (resulting line-width at half-height Δν½ was less than 3.5 Hz at 250 ms of acquisition time).

### EXAMPLES

### Preparation of Starting Materials

Zavegepant can be prepared according to methods known from the literature, for example U.S. Pat. No. 8,481,546 or according to International Publication No. WO 2022/217008.

Zavegepant hydrochloride may be prepared by converting Zavegepant prepared to general methods, such as those mentioned above, to a hydrochloride salt.

Amorphous Zavegepant hydrochloride may be prepared by any method, for example according to the example below:

### Preparation of Amorphous Zavegepant Hydrochloride:

Zavegepant hydrochloride (100 mg) was dissolved in ethanol (1 ml) at elevated temperature. The obtained clear solution was cooled down to a temperature of about 20° C. and heptane was added dropwise (300 μl). The product started to precipitate after a few minutes of stirring. The obtained suspension was stirred for 60 minutes. Then, the solid was filtered and dried under vacuum for about 15 minutes. The solid was analyzed by XRPD; amorphous was obtained.

### Example 1: Preparation of Zavegepant Hydrochloride Form C1

Zavegepant hydrochloride (amorphous, 500 mg) was slurried in methyl iso-butyl ketone (“MIBK”, 5 ml) for about 2 hours at a temperature of about 75° C. The obtained suspension was allowed to cool down to room temperature spontaneously. Then, the solid was filtered and dried under vacuum for about 15 minutes. The solid was analyzed by XRPD; Form C1 was obtained. An XRPD pattern is shown in FIG. 1.

### Example 2: Preparation of Zavegepant Hydrochloride Form C2

Zavegepant hydrochloride (amorphous, 5 grams) was slurried in acetonitrile (80 ml) for about 2.5 hours at a temperature of about 60° C. The obtained suspension was allowed to cool down to room temperature spontaneously, and was further maintained under stirring for a period of about 2 hours at room temperature. Then, the solid was filtered and dried under vacuum for about 1.5 hours. The solid was analyzed by XRPD; Form 2 was obtained. An XRPD pattern is shown in FIG. 2.

Further aspects and embodiments of the disclosure are set out in the following numbered clauses:

1. Crystalline Zavegepant hydrochloride form C2, which is characterized by an XRPD pattern having characteristic peaks at 12.6, 14.1, 14.9, 19.0, and 19.7 degrees 2-theta±0.2 degrees 2-theta.

2. Crystalline Zavegepant hydrochloride form C2 according to Clause 1, characterized by an XRPD pattern having characteristic peaks at 12.6, 14.1, 14.9, 19.0 and 19.7 degrees 2-theta±0.2 degrees 2-theta, and any one, two, three, four or five additional peaks at 9.8, 16.2, 17.6, 20.9 and 21.8 degrees 2-theta±0.2 degrees 2-theta.

3. Crystalline Zavegepant hydrochloride form C2 according to any of Clause 1 or Clause 2, characterized by an XRPD pattern having characteristic peaks at 9.8, 12.6, 14.1, 14.9, 16.2, 17.6, 19.0, 19.7, 20.9 and 21.8 degrees 2-theta±0.2 degrees 2-theta; or an XRPD pattern substantially as depicted in FIG. 2.

4. Crystalline Form C2 of Zavegepant hydrochloride according to any of Clauses 1, 2 and 3, which is further characterized by an X-ray powder diffraction pattern having an absence of peaks at 3.2 to 6.0 degrees 2-theta±0.2 degrees 2-theta.

5. Crystalline Form C2 of Zavegepant hydrochloride according to any of Clauses 1, 2 and 3, which is further characterized by an X-ray powder diffraction pattern having an absence of peaks at 3.2 to 6.5 degrees 2-theta±0.2 degrees 2-theta.

6. Crystalline Form C2 of Zavegepant hydrochloride according to any of Clauses 1, 2 and 3, which is further characterized by an X-ray powder diffraction pattern having an absence of peaks at 3.2 to 7.0 degrees±0.2 degrees 2-theta.

7. Crystalline Form C2 of Zavegepant hydrochloride according to any of Clauses 1, 2 and 3, which is further characterized by an X-ray powder diffraction pattern having an absence of peaks at 3.2 to 7.5 degrees 2-theta±0.2 degrees 2-theta.

8. Crystalline Form C2 of Zavegepant hydrochloride according to any of Clauses 1, 2 and 3, which is further characterized by an X-ray powder diffraction pattern having an absence of peaks at 3.2 to 8.0 degrees 2-theta±0.2 degrees 2-theta.

9. Crystalline Form C2 of Zavegepant hydrochloride according to any of Clauses 1, 2 and 3, which is further characterized by an X-ray powder diffraction pattern having an absence of peaks at 3.2 to 8.5 degrees 2-theta±0.2 degrees 2-theta.

10. Crystalline Form C2 of Zavegepant hydrochloride according to any of Clauses 1, 2 and 3, which is further characterized by an X-ray powder diffraction pattern having an absence of peaks at 3.2 to 9.0 degrees 2-theta±0.2 degrees 2-theta.

11. Crystalline Form C2 of Zavegepant hydrochloride according to any of Clauses 1, 2 and 3, which is further characterized by an X-ray powder diffraction pattern having an absence of peaks at 3.2 to 9.3 degrees 2-theta±0.2 degrees 2-theta.

12. Crystalline Form C2 of Zavegepant hydrochloride according to any of Clauses 1, 2, 3, 4, 5, 6, 7, 8, 9, 10, and 11, which is further characterized by an X-ray powder diffraction pattern having an absence of peaks at 11.5 to 12.1 degrees 2-theta±0.2 degrees 2-theta.

13. Crystalline Form C2 of Zavegepant hydrochloride according to any of Clauses 1, 2, 3, 4, 5, 6, 7, 8, 9. 10, 11, and 12, which is further characterized by an X-ray powder diffraction pattern having an absence of peaks at 16.7 to 16.8 degrees 2-theta±0.2 degrees 2-theta.

14. Crystalline Form C2 of Zavegepant hydrochloride according to any of Clauses 1, 2, 3, 4, 5, 6, 7, 8, 9, 10, 11, 12, and 13, and which is further characterized by an X-ray powder diffraction pattern having an absence of peaks at 18.1 to 18.5 degrees 2-theta±0.2 degrees 2-theta.

15. Crystalline Form C2 of Zavegepant hydrochloride according to any of Clauses 1, 2, 3, 4, 5, 6, 7, 8, 9, 10, 11, 12, 13, and 14, which is further characterized by a solid state ¹³C NMR spectrum having peaks in the range of 200-100 ppm at 170.2, 162.7, 158.4, 142.3 and 129.9 ppm±0.2 ppm.

16. Crystalline Form C2 of Zavegepant hydrochloride according to any of Clauses 1, 2, 3, 4, 5, 6, 7, 8, 9, 10, 11, 12, 13, 14, and 15, which is further characterized by a solid state ¹³C NMR spectrum having chemical shift absolute differences from reference peak at 123.5 ppm±1 ppm: 46.7; 39.2; 34.9; 18.8 and 6.5 ppm±0.1 ppm.

17. Crystalline Zavegepant hydrochloride form C2 according to any of Clauses 1, 2, 3, 4, 5, 6, 7, 8, 9, 10, 11, 12, 13, 14, 15, and 16, which is further characterized by a solid state ¹³C NMR spectrum having peaks at the range of 200-100 ppm at 170.2, 162.7, 158.4, 142.3, 139.2, 138.1, 135.1, 134.3, 131.1, 129.9, and 123.5 ppm±0.2 ppm.

18. Crystalline Zavegepant hydrochloride Form C2 according to any of Clauses 1, 2, 3, 4, 5, 6, 7, 8, 9, 10, 11, 12, 13, 14, 15, 16, and 17, which is further characterised by a solid state ¹³C NMR spectrum having peaks in the range of 200-100 ppm at: 170.2, 162.7, 158.4, 142.3, 139.2, 138.1, 135.1, 134.3, 131.1, 129.9, and 123.5 ppm±0.2 ppm, and additionally any one, two, three, or four peaks at: 122.1, 117.5, and 116.2 ppm±0.2 ppm.

19. Crystalline Zavegepant hydrochloride Form C2 according to any of Clauses 1, 2, 3, 4, 5, 6, 7, 8, 9, 10, 11, 12, 13, 14, 15, 16, 17, and 18, which is characterised by a solid state ¹³C NMR spectrum having peaks in the range of 200-100 ppm at: 170.2, 162.7, 158.4, 142.3, 139.2, 138.1, 135.1, 134.3, 131.1, 129.9, 123.5, 122.1, 117.5, and 116.2 ppm 0.2 ppm.

20. Crystalline Zavegepant hydrochloride Form C2 according to any of Clauses 1, 2, 3, 4, 5, 6, 7, 8, 9, 10, 11, 12, 13, 14, 15, 16, 17, 18, and 19, which is further characterized by DSC melting point is at about 261° C., preferably as measured by differential scanning calorimetry; or optionally by a DSC thermogram substantially as depicted in FIG. 4.

21. Crystalline Zavegepant hydrochloride form C2 according to any preceding clause, wherein it is an anhydrous form.

22. Crystalline Zavegepant hydrochloride form C2 according to any preceding clause, which contains: no more than about 20%, no more than about 10%, no more than about 5%, no more than about 2%, no more than about 1% or about 0% of any other crystalline forms of Zavegepant hydrochloride.

23. Crystalline Zavegepant hydrochloride form C2 according to any preceding clause, which contains: no more than about 20%, no more than about 10%, no more than about 5%, no more than about 2%, no more than about 1% or about 0% of amorphous Zavegepant hydrochloride.

24. Crystalline Zavegepant hydrochloride form C2 according to any preceding clause, which is chemically pure.

25. Crystalline Zavegepant hydrochloride Form C1, which is characterized by an XRPD pattern having characteristic peaks at 7.1, 9.4, 15.6, 17.5 and 21.2 degrees 2-theta±0.2 degrees 2-theta.

26. Crystalline Zavegepant hydrochloride Form C1 according to Clause 25, which is characterized by an XRPD pattern having characteristic peaks at 7.1, 9.4, 15.6, 17.5 and 21.2 degrees 2-theta±0.2 degrees 2-theta, and any one, two, three, four or five additional peaks at 10.6, 12.7, 14.7, 18.1 and 19.6 degrees 2-theta±0.2 degrees 2-theta.

27. Crystalline Zavegepant hydrochloride Form C1 according to any of Clause 25 or Clause 26, which is characterized by an XRPD pattern having characteristic peaks at 7.1, 9.4, 10.6, 12.7, 14.7, 15.6, 17.5, 18.1, 19.6 and 21.2 degrees 2-theta±0.2 degrees 2-theta or an XRPD pattern substantially as depicted in FIG. 1.

28. Crystalline Zavegepant hydrochloride Form C1 according to any of Clauses 25, 26 or 27, which is further characterized by an X-ray powder diffraction pattern having an absence of peaks at: 3.2 to 5.0 degrees 2-theta±0.2 degrees 2-theta.

29. Crystalline Zavegepant hydrochloride Form C1 according to any of Clauses 25, 26 or 27, which is further characterized by an X-ray powder diffraction pattern having an absence of peaks at 3.2 to 6.0 degrees±0.2 degrees 2-theta.

30. Crystalline Zavegepant hydrochloride Form C1 according to any of Clauses 25, 26 or 27, which is further characterized by an X-ray powder diffraction pattern having an absence of peaks at 3.2 to 6.3 degrees 2-theta±0.2 degrees 2-theta.

31. Crystalline Zavegepant hydrochloride Form C1 according to any of Clauses 25, 26, 27, 28, 29, or 30, which is further characterized by an X-ray powder diffraction pattern having an absence of peaks at 7.6 to 8.2 degrees 2-theta±0.2 degrees 2-theta.

32. Crystalline Zavegepant hydrochloride Form C1 according to any of Clauses 25, 26, 27, 28, 29, or 30, which further characterized by an X-ray powder diffraction pattern having an absence of peaks at 7.6 to 8.6 degrees 2-theta±0.2 degrees 2-theta.

33. Crystalline Zavegepant hydrochloride Form C1 according to any of Clauses 25, 26, 27, 28, 29, 30, 31, or 32, which is further characterized by an X-ray powder diffraction pattern having an absence of peaks at: 9.9 to 10.1 degrees 2-theta±0.2 degrees 2-theta.

34. Crystalline Zavegepant hydrochloride Form C1 according to any of Clauses 25, 26, 27, 28, 29, 30, 31, 32, or 33, which is further characterized by an X-ray powder diffraction pattern having an absence of peaks at: 11.1 to 11.7 degrees 2-theta±0.2 degrees 2-theta.

35. Crystalline Zavegepant hydrochloride Form C1 according to any of Clauses 25, 26, 27, 28, 29, 30, 31, 32, 33, or 34, which is further characterized by a DSC melting point (onset) at about 264° C. or a DSC thermogram substantially as depicted in FIG. 3.

36. Crystalline Zavegepant hydrochloride Form C1 according to any of Clauses 25, 26, 27, 28, 29, 30, 31, 32, 33, 34, or 35, which is isolated.

37. Crystalline Zavegepant hydrochloride Form C1 according to any of Clauses 25, 26, 27, 28, 29, 30, 31, 32, 33, 34, 35, or 36, which contains: about 20% (w/w) or less, about 10% (w/w) or less, about 5% (w/w) or less, about 2% (w/w) or less, about 1% (w/w) or less, or about 0% of any other crystalline forms of Zavegepant hydrochloride.

38. Crystalline Zavegepant hydrochloride Form C1 according to any of Clauses 25, 26, 27, 28, 29, 30, 31, 32, 33, 34, 35, 36, or 37, which contains: about 20% (w/w) or less, about 10% (w/w) or less, about 5% (w/w) or less, about 2% (w/w) or less, about 1% (w/w) or less, or about 0% of amorphous Zavegepant hydrochloride.

39. Crystalline Zavegepant hydrochloride Form C1 according to any of Clauses 25, 26, 27, 28, 29, 30, 31, 32, 33, 34, 35, 36, 37, or 38, which is substantially free of enantiomers of Zavegepant or Zavegepant hydrochloride, preferably containing: 20% (w/w) or less, about 10% (w/w) or less, about 5% (w/w) or less, about 2% (w/w) or less, about 1% (w/w) or less, or about 0% of other enantiomers of Zavegepant or Zavegepant hydrochloride respectively.

40. A pharmaceutical composition comprising a crystalline form according to any preceding clause.

41. A pharmaceutical formulation comprising a crystalline form according to any of Clauses 1 to 39, or a pharmaceutical composition of Clause 40, and at least one pharmaceutically acceptable excipient.

42. A pharmaceutical formulation according to Clause 41, optionally in the form of a liquid or suspension, and optionally wherein the formulation is an intranasal formulation.

43. A process for preparing a pharmaceutical formulation according to Clause 41 or Clause 42, comprising combining a crystalline form according to any of Clause 1 to 39, or a pharmaceutical composition of Clause 40, with at least one pharmaceutically acceptable excipient.

44. Use of a crystalline form according to any of Clause 1 to 39, for the preparation of a pharmaceutical composition and/or formulation, and optionally wherein the pharmaceutical formulation is a liquid or a suspension.

45. Use according to Clause 44, wherein the pharmaceutical composition or pharmaceutical formulation is an intranasal formulation.

46. A crystalline form according to any of Clause 1 to 39; a pharmaceutical composition according to Clause 40; or a pharmaceutical formulation according to Clause 41 or Clause 42, for use as a medicament.

47. A crystalline form according to any of Clause 1 to 39; a pharmaceutical composition according to Clause 40; or a pharmaceutical formulation according to Clause 41 or Clause 42, for use in the treatment or prevention of migraine; or COVID-19 respiratory infection.

48. A method of treating or preventing migraine; or COVID-19 respiratory infection, comprising administering a therapeutically effective amount of a crystalline form according to any of Clause 1 to 39; a pharmaceutical composition according to Clause 40; or a pharmaceutical formulation according to Clause 41 or Clause 42, to a subject in need of the treatment.

49. The method according to Clause 48, wherein the treatment is administrated intranasally.

50. A crystalline form according to any of Clause 1 to 39; a pharmaceutical composition according to Clause 40; or a pharmaceutical formulation according to Clause 41 or Clause 42, for the manufacture of a medicament for the treatment of migraine or COVID-19 respiratory infection.

51. Use of a crystalline form according to any of Clauses 1 to 39, in the preparation of another solid state forms of Zavegepant, Zavegepant salt, co-crystal or complex of Zavegepant or Zavegepant salt.

52. A process for preparing solid state form of Zavegepant, Zavegepant salt and their crystalline polymorph, co-crystals or complex thereof, comprising preparing a crystalline form according to any of Clauses 1 to 39, and converting it to another solid state form of Zavegepant, Zavegepant salt or a crystalline polymorph, co-crystals or complex thereof.

1. A crystalline Zavegepant hydrochloride form C2, which is characterized by an XRPD pattern having characteristic peaks at 12.6, 14.1, 14.9, 19.0 and 19.7 degrees 2-theta±0.2 degrees 2-theta.

2. The crystalline Zavegepant hydrochloride form C2 according to claim 1, characterized by an XRPD pattern having characteristic peaks at 12.6, 14.1, 14.9, 19.0 and 19.7 degrees 2-theta±0.2 degrees 2-theta, and any one, two, three, four or five additional peaks at 9.8, 16.2, 17.6, 20.9 and 21.8 degrees 2-theta±0.2 degrees 2-theta.

3. The crystalline Zavegepant hydrochloride form C2 according to claim 1, characterized by an XRPD pattern having characteristic peaks at 9.8, 12.6, 14.1, 14.9, 16.2, 17.6, 19.0, 19.7, 20.9 and 21.8 degrees 2-theta±0.2 degrees 2-theta; or an XRPD pattern substantially as depicted in FIG. 2.

4. The crystalline Zavegepant hydrochloride form C2 according to claim 1, which is further characterized by at least one of:
(a) a solid state ¹³C NMR spectrum having peaks at the range of 200-100 ppm at 170.2, 162.7, 158.4, 142.3 and 129.9 ppm±0.2 ppm; or
(b) a solid state ¹³C NMR spectrum having chemical shift absolute differences from reference peak at 123.5 ppm±1 ppm: 46.7, 39.2, 34.9, 18.8, and 6.5 ppm±0.1 ppm.

5. The crystalline Zavegepant hydrochloride form C2 according to claim 1, which is further characterized by a solid state ¹³C NMR spectrum having peaks at the range of 200-100 ppm at 170.2, 162.7, 158.4, 142.3, 139.2, 138.1, 135.1, 134.3, 131.1, 129.9, and 123.5 ppm±0.2 ppm.

6. The crystalline Zavegepant hydrochloride form C2 according to claim 1, which is anhydrous.

7. The crystalline Zavegepant hydrochloride form C2 according to claim 1, which contains no more than about 20%, no more than about 10%, no more than about 5%, no more than about 2%, no more than about 1% or about 0% of any other crystalline forms of Zavegepant hydrochloride.

8. The crystalline Zavegepant hydrochloride form C2 according to claim 1, which contains no more than about 20%, no more than about 10%, no more than about 5%, no more than about 2%, no more than about 1% or about 0% of amorphous Zavegepant hydrochloride.

9. The crystalline Zavegepant hydrochloride form C2 according to claim 1, which is chemically pure.

10. A pharmaceutical composition comprising the crystalline Zavegepant hydrochloride form C2 according to claim 1.

11. A pharmaceutical formulation comprising the crystalline Zavegepant hydrochloride form C2 according to claim 1 and at least one pharmaceutically acceptable excipient.

12. The pharmaceutical formulation according to claim 11, in the form of at least one of a liquid, a suspension, or an intranasal formulation.

13. A process for preparing a pharmaceutical formulation according to the process comprising combining the crystalline Zavegepant hydrochloride form C2 according to claim 1 with at least one pharmaceutically acceptable excipient.

14. (canceled)

15. (canceled)

16. The method according to claim 17, wherein the subject is in need of treatment or prevention of migraine or treatment of a COVID-19 respiratory infection.

17. A method of treatment comprising administering a therapeutically effective amount of the crystalline Zavegepant hydrochloride form C2 according to claim 1 to a subject in need of the treatment.

18. The method according to claim 17, wherein the treatment is administrated intranasally.

19. A method comprising: manufacturing a medicament for treatment of migraine or COVID-19 respiratory infection salt from the crystalline Zavegepant hydrochloride form C2 according to claim 1.

20. (canceled)

21. A process for preparing solid state form of Zavegepant, Zavegepant salt and their crystalline polymorph, co-crystals or complex thereof, comprising preparing the crystalline Zavegepant hydrochloride form C2 according to claim 1, and converting it to another solid state form of Zavegepant, Zavegepant salt or a crystalline polymorph, co-crystals or complex thereof.
